(19) United States
(12) Patent Application Publication
(10) Pub. No.: US 2015/0190568 A1
(43) Pub. Date: Jul. 9, 2015

(54) PRESSURE MANAGEMENT FOR IMPLANTABLE DRUG-DELIVERY DEVICES

(71) Applicants: Jason Shih, Yorba Linda, CA (US); Fukang Jiang, Arcadia, CA (US); Andrew Urazaki, Arcadia, CA (US)

(72) Inventors: Jason Shih, Yorba Linda, CA (US); Fukang Jiang, Arcadia, CA (US); Andrew Urazaki, Arcadia, CA (US)

(21) Appl. No.: 14/591,617

(22) Filed: Jan. 7, 2015

Related U.S. Application Data
(60) Provisional application No. 61/924,521, filed on Jan. 7, 2014.

(51) Int. Cl. A61M 5/142

(52) U.S. Cl. CPC A61M 5/14276; A61M 2005/14204; A61M 5/165

(57) ABSTRACT

Excess gas generated by and within electrolytic drug-pump devices is managed by strategically facilitating catalytic recombination or sequestration of excess electrolysis gases in void regions where such gases are most likely to accumulate.

### CROSS-REFERENCE TO RELATED APPLICATIONS

This application claims priority to and the benefit of U.S. Provisional Application No. 61/924,521, filed on Jan. 7, 2014, the entire disclosure of which is hereby incorporated by reference.

### BACKGROUND

Most drug-delivery devices utilize an actuation mechanism to drive medicament from a reservoir through a cannula into target areas. In general, pressurization occurs within the drug-delivery device or at the interface between the device and its surroundings. The pressure magnitudes and gradients in these regions can make it difficult to precisely control delivery of small amounts of drug, especially when the device is refillable or used for repeated dosing over a relatively long term. For example, without proper regulation of the pressure in the drug reservoir, pressure or vacuum buildup can interfere with smooth, continuous administration of a liquid medicament. This problem is particularly acute in devices whose driving mechanism involves generation of pressurized gas; in such devices, excess gas can be generated and may leak to various device regions. In addition, when the device is implanted in a patient, the difficulties of limited physical space and access to the device, as well as the overall complexity of in vivo implantation and operation, can make pressure regulation in the device challenging.

Excess gas can also adversely affect refilling. As gas accumulates in “headspace”—i.e., unfilled volume within the sealed drug-delivery device—it can complicate the refilling process and create considerable dead (i.e., unusable) interior volume. More importantly, a variety of drug-delivery devices have compliant reservoir walls to minimize dead volumes and provide ease in handling during refilling. With these devices, excess gas accumulating in peripheral regions creates a differential pressure that can eventually prevent the refilling operation from proceeding to completion.

Managing the buildup of excess gas usually involves ensuring gas-tightness along the pressurization path. This can require significant efforts in design, manufacture, and quality control. For example, in electrolytic drug-delivery devices, hydrogen and oxygen are generated as an actuating medium during dosing. Hydrogen is known to penetrate thin walls and peripheral edges over time, and so may leak into reservoir chambers and escape through their perimeters. Over extended operating time frames (e.g., years), these gases may accumulate in pump headspace regions and interfere with dosing and refill operations absent corrective measures.

### SUMMARY

In various embodiments, the present invention relates to management of excess gas generated by and within electrolytic drug-pump devices. In particular, catalytic recombination of excess electrolysis gases is strategically facilitated in void regions where gases are most likely to accumulate.

Accordingly, in a first aspect, the invention pertain to an implantable pump comprising a drug reservoir, a cannula, an electrolytic pumping mechanism for forcing liquid from the reservoir through the cannula, a shell at least partly surrounding the drug reservoir and the pumping mechanism in spaced-apart relation therefrom to create an interior headspace volume, and a recombination catalyst disposed within the headspace volume to cause recombination of electrolysis gas therein. In some embodiments, the pumping mechanism comprises an electrolysis chamber formed by a floor and, thereover, an expandable membrane fastened to the floor along a peripheral edge of the membrane, and within the electrolysis chamber, a plurality of electrolysis electrodes, and the drug reservoir is formed by the expandable membrane and, thereover, a flexible dome fastened to at least one of the floor or the expandable membrane along a peripheral edge of the dome, whereby the membrane provides a fluid barrier between the electrolysis chamber and the drug reservoir and expansion of the membrane reduces a volume of the drug reservoir. In such embodiments, the shell may have an inner surface facing an outer surface of the rigid dome, and the recombination catalyst may be physically associated with at least a portion of at least one of the inner shell surface or the outer dome surface. By “physically associated” is meant affixed to or deposited onto the interior wall, or physically integrated with or within the wall so as to present a surface to the headspace volume. Alternatively or in addition, the recombination catalyst may be physically associated with the peripheral edge of the dome within the headspace and/or with a peripheral edge of the shell within the headspace.

In various embodiments, the pump further comprises resealable fluid port in the shell; the port is normally sealed and facilitates fluidic access to the headspace. In some embodiments, the catalyst consists essentially of platinum. The pump may also contain, within the headspace, at least one liquid getter and/or at least one gas getter.

In another aspect, the invention relates to a method of operating an implantable pump comprising a drug reservoir, a cannula, an electrolytic pumping mechanism for forcing liquid from the reservoir through the cannula, a shell at least partly surrounding the drug reservoir and the pumping mechanism in spaced-apart relation therefrom to create a headspace volume, and a recombination catalyst disposed within the headspace volume. In various embodiments, the method comprises the steps of operating the pumping mechanism whereby electrolysis gas components enter the headspace and are caused to recombine into a liquid by the recombination catalyst therein, and periodically emptying the headspace of accumulated liquid.

During the emptying step, the headspace may in some embodiments, be accessed through a resealable fluid port in the shell. In various embodiments, (i) the pumping mechanism comprises an electrolysis chamber formed by a floor and, thereover, an expandable membrane fastened to the floor along a peripheral edge of the membrane, and within the electrolysis chamber, a plurality of electrolysis electrodes, and (ii) the drug reservoir is formed by the expandable membrane and, thereover, a flexible dome fastened to at least one of the floor or the expandable membrane along a peripheral edge of the dome, whereby the membrane provides a fluid barrier between the electrolysis chamber and the drug reservoir and expansion of the membrane reduces a volume of the drug reservoir. The may have an inner surface facing an outer surface of the rigid dome, and the recombination catalyst may be physically associated with at least a portion of at least one of the inner shell surface or the outer dome surface. Alternatively or in addition, the recombination catalyst may be physically associated with the peripheral edge of the dome within the headspace and/or with a peripheral edge of the shell within the headspace. The catalyst may consist essentially of platinum. In some embodiments, the electrolytic pumping mechanism is actuated to apply pressure to the headspace in conjunction with the periodic emptying step.

In still another aspect, the invention relates to an implantable pump comprising a drug reservoir, a cannula, an electrolytic pumping mechanism for forcing liquid from the reservoir through the cannula, a shell at least partly surrounding the drug reservoir and the pumping mechanism in spaced-apart relation therefrom to create an interior headspace volume, and a liquid getter and/or a gas getter disposed within the headspace volume to cause sequestration of electrolysis gas therein.

In still another aspect, the invention pertains to a method of operating an implantable pump comprising a drug reservoir, a cannula, an electrolytic pumping mechanism for forcing liquid from the reservoir through the cannula, a shell at least partly surrounding the drug reservoir and the pumping mechanism in spaced-apart relation therefrom to create a headspace volume, and at least one of a liquid getter or a gas getter disposed within the headspace volume. In various embodiments, the method comprises the steps of operating the pumping mechanism whereby electrolysis gas components enter the headspace and are caused to recombine into a liquid by the recombination catalyst therein, and periodically emptying the headspace of spent getter (e.g., a sequestration agent that has reached its capacity).

The term “substantially” or “approximately” means ±10% (e.g., by weight or by volume), and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

The foregoing will be more readily understood from the following detailed description of the invention, in particular, when taken in conjunction with the drawings, in which:

FIG. 1 is a sectional view of an electrolytic drug pump showing the operative components thereof.

FIG. 2 is a partial section of a drug pump incorporating a rigid shell, with the operative components not shown.

### DETAILED DESCRIPTION

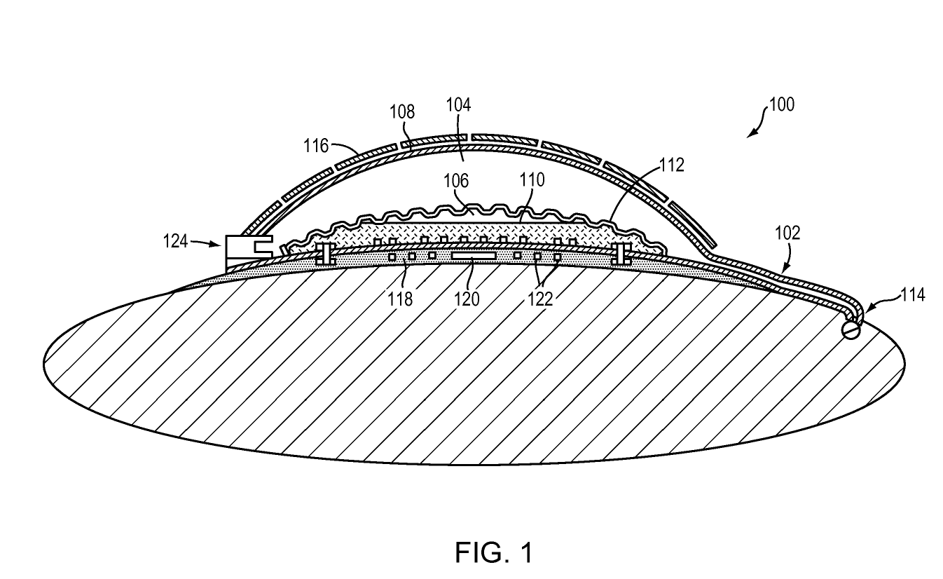

FIG. 1 illustrates an exemplary drug-delivery pump implanted within a patient's eye. It should be understood, however, that many features relevant to ophthalmic pumps are also applicable to other drug-pump devices, such as, e.g., implantable pain pumps and insulin pumps. Accordingly, where reference to the eye is made in the following description, or in the figures, such reference is generally intended to be merely illustrative, and not as limiting the scope of the invention.

The device 100 includes a cannula 102 and a pair of chambers 104, 106 bounded by a first envelope 108. The top chamber 104 defines a drug reservoir that contains the drug to be administered in liquid form, and the bottom chamber 106 contains a liquid which, when subjected to electrolysis using electrolysis electrodes 110, evolves a gaseous product. In one embodiment, the electrolysis electrodes 110 are platinum. Alternatively, any other appropriate conductive material (e.g., copper, gold, or silver on parylene, ceramic, or a biocompatible insulator) may be used. Additional catalyst elements (e.g., constructed from platinum) may be located within the electrolysis chamber 106 to act as a recombination catalyst to encourage the phase change of the electrolyte from its gaseous state to its liquid state when the electrolysis electrodes 110 are turned off. The electrolyte fluid contained within the electrolysis chamber 104 may be a saline (i.e., NaCl and H₂O) solution, or a solution that contains either magnesium sulfate or sodium sulfate, or may be pure water or any non-toxic solution. The two chambers 104, 106 are separated by an expandable (e.g., corrugated) diaphragm 112. The envelope 108 and diaphragm 112 may be made of a biocompatible polymeric material, e.g., parylene.

The cannula 102 connects the top drug chamber 104 with a check valve 114 inserted at the site of administration or anywhere along the fluid path between the drug reservoir and site of administration. The envelope 108 may reside within a shaped protective shell 116 made of a flexible material (e.g., a bladder or collapsible chamber) or a relatively rigid biocompatible material (e.g., medical-grade polypropylene). Control circuitry 118, a battery 120, and an induction coil 122 for power and data transmission are embedded between the bottom wall of the electrolyte chamber 106 and the floor of the shell 116. Depending on the complexity of the control functionality it provides, the control circuitry 118 may be implemented, e.g., in the form of analog circuits, digital integrated circuits (such as, e.g., microcontrollers), or programmable logic devices. In some embodiments, the control circuitry 118 includes a microprocessor and associated memory for implementing complex drug-delivery protocols. The induction coil 122 may be located elsewhere for improved data and power communication and connected to the control circuitry 118. The drug pump device 100 may also include various sensors (e.g., pressure and flow sensors) for monitoring the status and operation of the various device components, and such data may be logged in the memory for subsequent retrieval and review.

Importantly for the prolonged use of the drug pump device 100 following implantation, the device 100 includes one or more ports 124 in fluid communication at least with the drug reservoir 104, which permit a refill needle (not shown) to be inserted therethrough. The ports may be polymer tubes, metal vias through wafers, pierceable septums, or other fluidic connections that include a check valve or equivalent functionality.

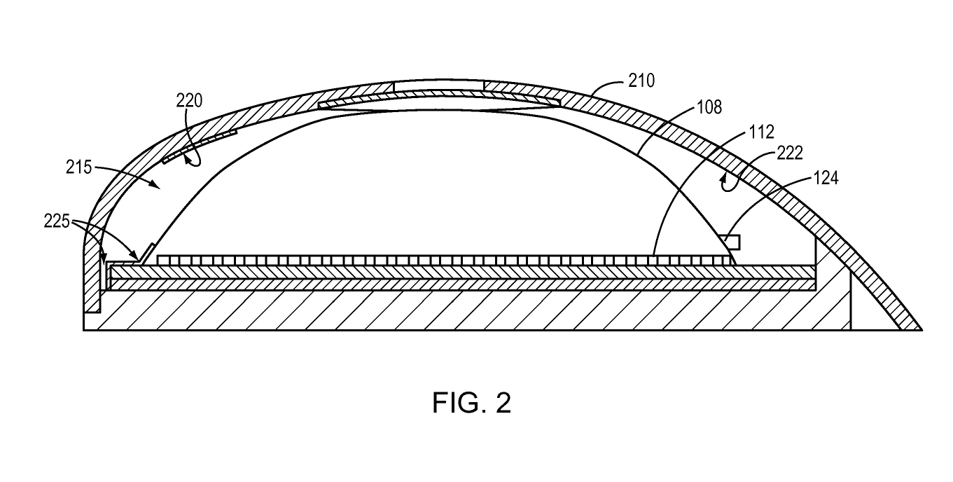

In various practical implementations, the outer shell 116 does not tightly conform to the envelope 108. Instead, as shown in FIG. 2, a hard outer shell 210 generally surrounds the operative components of the pump but has a different shape conforming, for example, to the site of implantation. The shell 210 may be made of, e.g., titanium. As a consequence of the difference in shape between the envelope 108 and the the outer shell 210, an enclosed headspace region 215 is created therebetween. Although not shown in FIG. 2 for simplicity of presentation, the port 124 is accessible through the shell 210 and extends into the reservoir 104. For example, the port 124 may have a septum that admits a refill needle but reseals following withdrawal of the needle; the septum may have a slit that is normally closed due to the elastomeric nature of the septum and, optionally, radial forces of confinement. As explained below, the shell 210 may have one or more additional ports to facilitate access to the headspace region 215.

The shell 210 provides a substantially hermetic seal to protect electronics, drugs, and other device components from coming in contact with bodily fluids. In typical implementations, the envelope 108 is polymeric and at least somewhat flexible. As a result, pressure accumulating in the headspace 215 as a consequence of the hermetic seal acts against the envelope 108 and effectively reduces the volume of the drug reservoir 104. At the same time, the accumulating gas reduces the vacuum necessary to aspirate the drug reservoir. In particular, the vacuum required to aspirate a given volume of liquid from the drug reservoir is given by

$${P}_{vac} = \frac{{P}_{at m} \times {V}_{head}}{{V}_{head} + {V}_{asp}}$$

where P_{vac} is the necessary vacuum level, Pₐₜₘ is atmospheric pressure at sea level (i.e., 14.7 psia), V_{head} is the original headspace volume, and Vₐₛₚ is the volume of liquid to be aspirated.

Thus, while the accumulated gases in the headspace may advantageously reduce the vacuum pressure required to aspirate the drug reservoir 104 and the pressure needed to expel the drug, it will simultaneously, and disadvantageously, reduce the amount of material that the drug reservoir 104 can contain. This, in turn, alters the interaction between the drug reservoir 104 and any downstream check valves, which may lead to unintentional drug delivery.

To mitigate the effects of gas permeation into the headspace, strategies for causing recombination of electrolysis gas components are employed. For example, some or all of the interior surfaces defining the headspace 215 can be coated with a recombination-promoting catalyst (e.g., platinum). Platinum may be coated globally over the entire interior surface or strategically introduced onto specific regions within the headspace 215. For example, a catalyst 220 may be coated onto all or a portion of the interior surface 222 of the shell 210. Alternatively or in addition, the catalyst may be applied to area around the port 124, as well as to the perimeter of envelope 108 and/or the perimeter of the shell 210 (as indicated at 225).

While the slow accumulation of liquid deposited in the headspace by recombining electrolysis gases may be insignificant for some time, it may be advantageous to include one or more resealable ports to the headspace 215 to permit withdrawal of accumulated liquid therefrom. It should be noted that the seal created by the shell 210 is usually not perfectly hermetic due to the various fluid pathways through the headspace 215, but the headspace should not have any direct liquid or gas leak; the majority of headspace liquid will therefore arise from the recombination of electrolysis gases therein. To promote the complete removal of liquid and gas from the headspace, the pumping mechanism (i.e., the electrolysis engine) may be actuated to apply pressure to the headspace in conjunction with any external actuation (e.g., a vacuum pump utilized to draw out the contents of the headspace.)

In some embodiments where the ratio of gases that accumulate in the headspace do not allow for liquid accumulation (e.g., too much hydrogen and too little oxygen), additional materials (e.g., “getters” that selectively sequester particular types of gas and/or liquid) are introduced into the headspace. Liquid getters (e.g., water-vapor scavengers such as titanium, zirconium, combinations thereof and other known materials) may be built into the shell or disposed on its interior surface to create sufficient sorption of water to negate the effect on the pump's functionality. Built-in gas getters (e.g. metal hydrides such as ZrAl₂) may be used to absorb particular gases that may accumulate to create non-negligible pressures affecting pump functionality as described above. As with all getters, the reactive surface area will be determined by estimated gas specific permeation rates.

Periodic emptying may be carried out so as to accommodate specific ratios of gases. Where there is a quantity of hydrogen gas that does not recombine due to an insufficient quantity of oxygen gas, for example, emptying may occur by drawing a vacuum to remove the excess hydrogen gas, or by injecting a complementary gas such as oxygen to facilitate recombination (and, e.g., removal or absorption by implanted getters to negate any material effect on pump functionality), followed by liquid removal. After emptying, a small volume of the complementary recombination gas may be injected into the headspace to allow for adequate recombination with the hydrogen that accumulates until the next periodic emptying of the liquid. Spent getters may also be removed and replaced.

Having described certain embodiments of the invention, it will be apparent to those of ordinary skill in the art that other embodiments incorporating the concepts disclosed herein may be used without departing from the spirit and scope of the invention. For example, various features described with respect to one particular device type and configuration may be implemented in other types of devices and alternative device configurations as well. Accordingly, the described embodiments are to be considered in all respects as only illustrative and not restrictive.

What is claimed is:

1. An implantable pump comprising:
a drug reservoir;
a cannula;
an electrolytic pumping mechanism for forcing liquid from the reservoir through the cannula;
a shell at least partly surrounding the drug reservoir and the pumping mechanism in spaced-apart relation therefrom to create an interior headspace volume; and
a recombination catalyst disposed within the headspace volume to cause recombination of electrolysis gas therein.

2. The pump of claim 1, wherein (i) the pumping mechanism comprises an electrolysis chamber formed by a floor and, thereover, an expandable membrane fastened to the floor along a peripheral edge of the membrane, and within the electrolysis chamber, a plurality of electrolysis electrodes, and (ii) the drug reservoir is formed by the expandable membrane and, thereover, a flexible dome fastened to at least one of the floor or the expandable membrane along a peripheral edge of the dome, whereby the membrane provides a fluid barrier between the electrolysis chamber and the drug reservoir and expansion of the membrane reduces a volume of the drug reservoir.

3. The pump of claim 2, wherein the shell has an inner surface facing an outer surface of the rigid dome, the recombination catalyst being physically associated with at least a portion of at least one of the inner shell surface or the outer dome surface.

4. The pump of claim 2, wherein the recombination catalyst is physically associated with the peripheral edge of the dome within the headspace.

5. The pump of claim 2, wherein the recombination catalyst is physically associated with a peripheral edge of the shell within the headspace.

6. The pump of claim 1, further comprising resealable fluid port in the shell, the port being normally sealed and facilitating fluidic access to the headspace.

7. The pump of claim 1, wherein the catalyst consists essentially of platinum.

8. The pump of claim 1, further comprising, within the headspace, at least one liquid getter.

9. The pump of claim 1, further comprising, within the headspace, at least one gas getter.

10. A method of operating an implantable pump comprising a drug reservoir, a cannula, an electrolytic pumping mechanism for forcing liquid from the reservoir through the cannula, a shell at least partly surrounding the drug reservoir and the pumping mechanism in spaced-apart relation therefrom to create a headspace volume, and a recombination catalyst disposed within the headspace volume, the method comprising the steps of:
operating the pumping mechanism whereby electrolysis gas components enter the headspace and are caused to recombine into a liquid by the recombination catalyst therein; and
periodically emptying the headspace of accumulated liquid.

11. The method of claim 10, wherein during the emptying step, the headspace is accessed through a resealable fluid port in the shell.

12. The method of claim 10, wherein (i) the pumping mechanism comprises an electrolysis chamber formed by a floor and, thereover, an expandable membrane fastened to the floor along a peripheral edge of the membrane, and within the electrolysis chamber, a plurality of electrolysis electrodes, and (ii) the drug reservoir is formed by the expandable membrane and, thereover, a flexible dome fastened to at least one of the floor or the expandable membrane along a peripheral edge of the dome, whereby the membrane provides a fluid barrier between the electrolysis chamber and the drug reservoir and expansion of the membrane reduces a volume of the drug reservoir.

13. The method of claim 12, wherein the shell has an inner surface facing an outer surface of the rigid dome, the recombination catalyst being physically associated with at least a portion of at least one of the inner shell surface or the outer dome surface.

14. The method of claim 12, wherein the recombination catalyst is physically associated with the peripheral edge of the dome within the headspace.

15. The method of claim 12, wherein the recombination catalyst is physically associated with a peripheral edge of the shell within the headspace.

16. The method of claim 10, wherein the catalyst consists essentially of platinum.

17. The method of claim 9, wherein the electrolytic pumping mechanism is actuated to apply pressure to the headspace in conjunction with the periodic emptying step.

18. An implantable pump comprising:
a drug reservoir;
a cannula;
an electrolytic pumping mechanism for forcing liquid from the reservoir through the cannula;
a shell at least partly surrounding the drug reservoir and the pumping mechanism in spaced-apart relation therefrom to create an interior headspace volume; and
at least one of a liquid getter or a gas getter disposed within the headspace volume to cause sequestration of electrolysis gas therein.

19. A method of operating an implantable pump comprising a drug reservoir, a cannula, an electrolytic pumping mechanism for forcing liquid from the reservoir through the cannula, a shell at least partly surrounding the drug reservoir and the pumping mechanism in spaced-apart relation therefrom to create a headspace volume, and at least one of a liquid getter or a gas getter disposed within the headspace volume, the method comprising the steps of:
operating the pumping mechanism whereby electrolysis gas components enter the headspace and are caused to recombine into a liquid by the recombination catalyst therein; and
periodically emptying the headspace of spent getter.
